(19) United States
(12) Patent Application Publication
(10) Pub. No.: US 2026/0130781 A1
(43) Pub. Date: May 14, 2026

(54) APPARATUS FOR BRACE AND CRYOTHERAPY

(71) Applicant: Eugene Luze, Highmore, SD (US)

(72) Inventor: Eugene Luze, Highmore, SD (US)

(21) Appl. No.: 18/941,389

(22) Filed: Nov. 8, 2024

(51) Int. Cl. A61F 5/01; A61F 7/00

(52) U.S. Cl. CPC A61F 5/0125; A61F 7/00; A61F 2007/0042; A61F 2007/0056; A61F 2007/0094

(57) ABSTRACT

An apparatus and a method for supporting a body part by limiting the movement of the body part. The apparatus also allows for providing cryotherapy to the body part. For example, the body part can be a knee joint, and the apparatus can be worn over the knee allowing flexing of the knee but limiting any rotational movement. The apparatus includes a brace for the support and a cooling bladder integrated with the brace for the cryotherapy. Cooling liquid can be circulated through the cooling bladder to extract heat from the body part. The cooling bladder is positioned inside of the brace so that to contact the skin.

### FIELD OF INVENTION

This present invention relates to a brace for treatment for an injury, post-operative care or both, and more particularly, the present invention relates to a brace for supportive care and cryotherapy.

### BACKGROUND

Cold compression therapy is used for healing and recovery after a patient has undergone orthopedic surgery or is recovering from an injury. Typically, cold compression devices are available for cryotherapy in which cold water cycles through a wrap that envelopes the surgical site. To put on the cold compression device, patients need to take off any hard brace worn for support and protection. This can be a difficult and painful task, especially for people who do not have assistance in accomplishing this task. The strain of having to swap between a hard brace and a cold compression device can also aggravate the surgery site, prolonging the recovery process. In many cold compression devices commercially available, the water does not stay cold for a prolonged period. The cold compression devices require period refilling of the reservoir with ice water during use. Also, the device needs a refill with cold water to start the therapy.

Changing water in between a therapy has a major drawback, i.e., ceasing the operation of the device to replenish the ice water interrupts the healing process. Interruptions are not desired because of the negative impacts on the outcome of cryotherapy and patient compliance.

An apparatus that can overcome the aforesaid problems is a long-term and unsolved need. An apparatus is needed that does not require removing a brace before cold therapy and interruptions.

### SUMMARY OF THE INVENTION

The following presents a simplified summary of one or more embodiments of the present invention to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to an apparatus that can provide protective support to a body part like a brace and also cryotherapy.

Another object of the present invention is that the apparatus does not need any modification for cryotherapy.

Still, another object of the present invention is that there is no need for refilling cold water.

Yet another object of the present invention is that the apparatus is easy to use.

A further object of the present invention is that a single apparatus is used for both support and cryotherapy preventing any need to switch between devices.

Still, a further object of the present invention is that the temperature in cryotherapy can be controlled.

Yet a further object of the present invention is that the efficacy of cryotherapy can be improved.

An additional object of the present invention is that cryotherapy can be controlled by an external computing device.

In one aspect, disclosed is an apparatus for supporting a body part and cryotherapy, the apparatus comprises a brace for supporting the body part, wherein the brace is configured to wrap around the body part and limit movement of the body part. The apparatus further comprises a cooling bladder coupled to the brace, wherein the cooling bladder is configured to receive cooling liquid for cryotherapy. The brace comprises an upper member and a lower member, the upper member comprises an upper left bracket and an upper right bracket, the lower member comprises a lower left bracket and a lower right bracket, wherein the cooling bladder is attached to the upper right bracket, the lower left bracket, and the lower right bracket. The upper left bracket and the lower left bracket are connected through a left hinge joint, and the upper right bracket and the lower right bracket are connected through a right hinge joint. The upper left bracket and the upper right bracket are connected through a first strap, wherein the lower left bracket and the lower right bracket are connected through a second strap.

In one aspect, each of the upper left bracket, the upper right bracket, the lower left bracket, and the lower right bracket comprises a top adapter plate and a bottom adapter plate, wherein portions of the cooling bladder are sandwiched between the top and bottom adapter plates of each of the upper left bracket, the upper right bracket, the lower left bracket, and the lower right bracket.

In one aspect, the cooling bladder comprises an inlet port, an internal volume, and an outlet port, wherein the inlet port and the outlet port are in fluid communication through the internal volume such as the cooling liquid is received through the inlet port circulates through the internal volume and exits from the outlet port.

In one aspect, the apparatus further comprises a cooling unit, wherein the inlet port and the outlet port of the cooling bladder are configured to fluidly couple to the cooling unit through tubing, wherein the cooling unit is configured to cool the cooling liquid and circulate the cooling liquid through the cooling bladder.

In one aspect, the apparatus further comprises a control unit configured to control the cooling unit for cryotherapy.

In one aspect, the body part is a knee, and the brace is a knee brace, wherein the upper member is configured to be wrapped around a thigh, and the lower member is configured to be wrapped around a calf.

In one aspect, disclosed is a method for supporting a body part and providing cryotherapy to the body part while supporting the body part, the method comprises providing a brace configured to be wrapped around the body part for supporting the body part by limiting the movement of the body part; and coupling a cooling bladder to an inner side of the brace, wherein the cooling bladder is configured to receive cooling liquid for the cryotherapy. The brace comprises an upper member and a lower member, the upper member comprises an upper left bracket and an upper right bracket, the lower member comprises a lower left bracket and a lower right bracket, wherein the cooling bladder is coupled to the upper right bracket, upper lower bracket, the lower left bracket, and the lower right bracket. The upper left bracket and the lower left bracket are connected through a left hinge joint, and the upper right bracket and the lower right bracket are connected through a right hinge joint. The upper left bracket and the upper right bracket are connected through a first strap, wherein the lower left bracket and the lower right bracket are connected through a second strap.

In one aspect, each of the upper left bracket, the upper right bracket, the lower left bracket, and the lower right bracket comprises a top adapter plate and a bottom adapter plate, wherein the method comprises sandwiching portions of the cooling bladder between the top and bottom adapter plates of each of the upper left bracket, the upper right bracket, the lower left bracket, and the lower right bracket for coupling the cooling bladder to the brace.

In one aspect, the cooling bladder comprises an inlet port, an internal volume, and an outlet port, wherein the inlet port and the outlet port are in fluid communication through the internal volume such as the cooling liquid is received through the inlet port circulates through the internal volume and exits from the outlet port. The apparatus further comprises a cooling unit, wherein the inlet port and the outlet port of the cooling bladder are configured to fluidly couple to the cooling unit through tubing, wherein the cooling unit is configured to cool the cooling liquid and circulate the cooling liquid through the cooling bladder. The apparatus further comprises a control unit configured to control the cooling unit for cryotherapy.

In one aspect, the body part is a knee, and the brace is a knee brace, wherein the method further comprises securing the upper member around a thigh; securing the lower member around a calf; connecting the cooling bladder to the cooling unit through the tubing; circulating the cooling liquid through the cooling bladder for a predetermined duration for the cryotherapy; and after the predetermined duration, turning off the cooling unit and disconnecting the cooling bladder from the cooling unit.

### BRIEF DESCRIPTION OF DRAWINGS

The accompanying figures, which are incorporated herein, form part of the specification and illustrate embodiments of the present invention. Together with the description, the figures further explain the principles of the present invention and enable a person skilled in the relevant arts to make and use the invention.

FIG. 1 is a block diagram showing the architecture of the apparatus, according to an exemplary embodiment of the present invention.

FIG. 2 shows a brace of the apparatus, according to an exemplary embodiment of the present invention.

FIG. 3 shows a cooling bladder of the apparatus, according to an exemplary embodiment of the present invention.

FIG. 4 shows a schematic diagram of the cooling bladder, according to an exemplary embodiment of the present invention.

FIG. 5 shows the combination of the cooling bladder and the brace, according to an exemplary embodiment of the present invention.

FIG. 6 shows a block diagram of a cooling unit of the apparatus, according to an exemplary embodiment of the present invention.

FIG. 7 shows a person wearing a knee brace and a colling unit of the apparatus, according to an exemplary embodiment of the present invention.

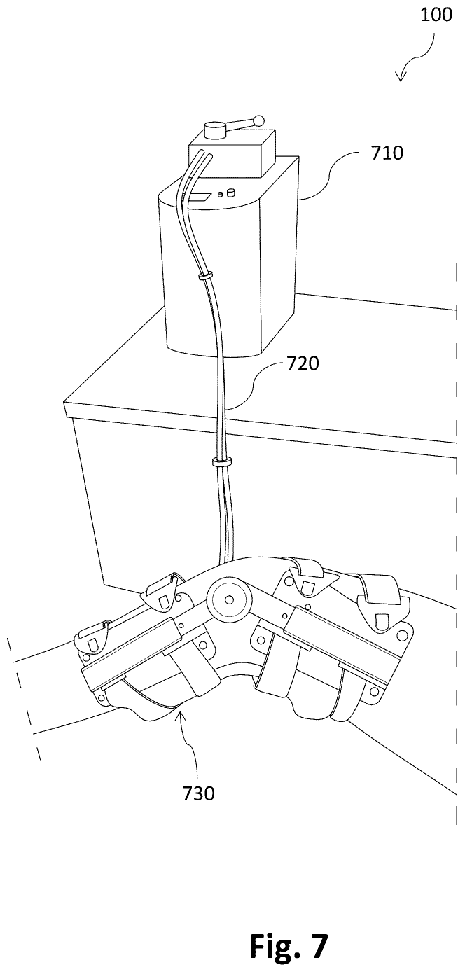

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

Disclosed is an apparatus for the protection and supportive care of a body part, such as a joint. The apparatus can be used in post-operative care to limit the movement of a joint. The disclosed apparatus can also be used for cryotherapy. For example, after knee surgery, the disclosed apparatus can be used for protecting, supporting, and limiting movement of the knee, like a knee brace. However, unlike a conventional knee brace, the disclosed apparatus can also provide cryotherapy without the need to remove the apparatus from the knee joint.

The apparatus includes a knee brace that can be worn around a knee for protecting, supporting, and/or limiting the movement of the knee. The knee brace helps to maintain the alignment of the knee joint during movement. The disclosed apparatus can be used in post-operative care of the knee or any knee disorder. The disclosed apparatus further includes an integrated cold compression wrap, also referred to herein as a cooling bladder. The cold compression wrap can be in contact with the skin or at least at some distance from the skin for the removal of heat from the skin. The integrated cold compression wrap allows for cryotherapy without removing the knee brace. Thus, the disclosed apparatus enhances the postoperative recovery process for orthopedic surgery patients.

It is to be understood that the above embodiments describe the apparatus including a knee brace for knee joints, however, the apparatus may include braces for different joints in the body, such as ankle joint and elbow joint. The integration of cold compression wrap streamlines the use of this product, making it more convenient and less painful.

The apparatus may include a cooling unit that may use a suitable compressor and refrigerants for cryotherapy. The refrigerant may also be referred to herein as a cooling liquid. The cooling liquid can run through the cold compression wrap to remove heat. The use of a cooling unit according to the present invention eliminates the need for refilling ice water in the beginning or during therapy. This can be particularly useful for effective therapy without any interruption and nuisance of changing the ice water. Instead of using refrigeration components, a Peltier device can also be used. The cooling unit may allow maintaining a low temperature in the cold compression wrap, such as 38° F.

The apparatus may also include a control unit that can be used to configure the temperature and duration of the cryotherapy. The automation of cryotherapy by the control unit can ensure patient safety and comfort. The control unit can be operated by an external computing device, such as a smartphone, desktop computer, laptop, smartwatch, tablet computer, and the like. An interface can be provided on the external computing device that allows for interaction with the control unit.

The control unit and the external computing unit can be connected through a suitable wired or wireless network. Examples of the wireless network include Bluetooth®, Wi-Fi, and the like. Through an external computing device, a user can select a profile from one or more profiles, wherein each profile may include different parameters of the cryotherapy. The user may also be provided with an option to create and save custom profiles. Also, the user can just enter the temperature and turn on and off the cooling unit. It is to be noted that a control button and display can be provided on the control unit for selecting temperatures, duration, turning the cooling unit on and off, and the like parameters.

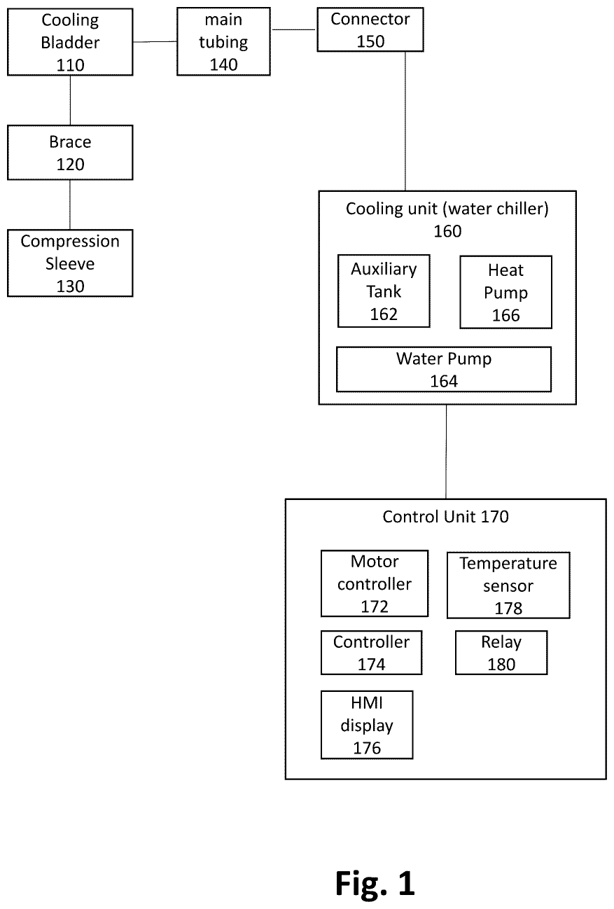

Referring to FIG. 1, which is a block diagram showing the architecture of the disclosed apparatus. The apparatus may include a brace 120, such as, but not limited to a knee brace that can be used to support a joint and protect the same. The brace can limit the movement of the joint and prevent any undesired alignment or movement. A cooling bladder 110 may be integral with the brace. The cooling bladder may also be removed and replaced with the same or a new cooling bladder. The apparatus may also include a compression sleeve 130. A water chiller 160 can provide cool water for cryotherapy. The water chiller allows circulating cool water through the cooling bladder. The water chiller 160 may include an auxiliary tank 162, a water pump 164, and a heat pump 166. The apparatus may also include a control unit 170 for controlling the operations of the cryotherapy. The control unit 170 may include a motor controller 172, a controller 174, an HMI display 176, a temperature sensor 178, and a relay 180.

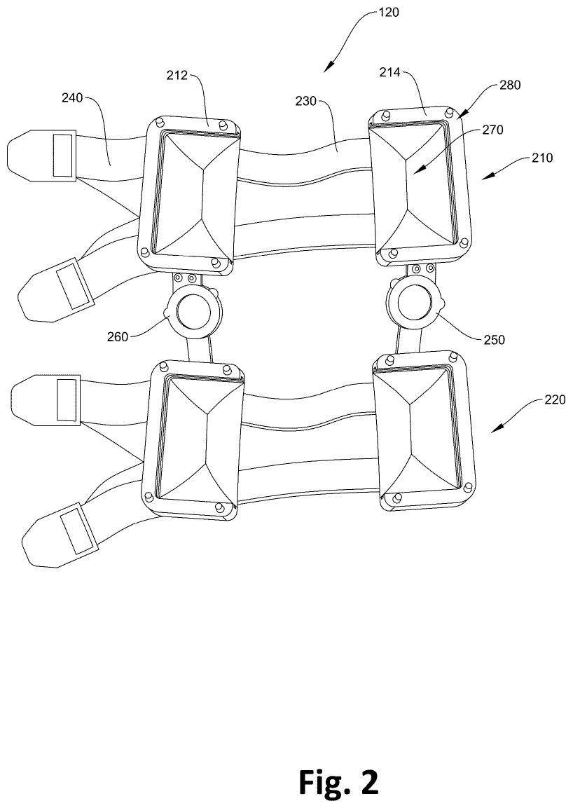

FIG. 2 shows a perspective view of the brace 120. The brace can limit the movement of the joint and prevent any undesired alignment or movement. The brace includes an upper member 210 and a lower member 220, wherein the upper member can be worn around the thigh and the lower member can be worn around the calf, i.e., opposite sides of a knee joint. Each of the upper member 210 and the lower member 220 includes a left bracket and a right bracket. FIG. 2 shows the upper member 210 may have a left bracket 212 and a right bracket 214. The left and right brackets are connected through a pair of straps 230. Fastening straps 240 also extend between the left and right brackets of each of the upper and lower members. The left brackets of the upper and lower members may be connected through a first hinge joint 250. Similarly, the right brackets of the upper and lower members may be connected through a second hinge joint 260. The hinge joints allow guided flexing of the knee joint limiting the rotation of the knee. Each bracket includes a top adapter plate and a bottom adapter plate. FIG. 2 shows the bottom adapter plates of each bracket. Each of the top and bottom adapter plates includes a central depression 270 and a flange 280 along three sides of the central depression 270. The brackets can be made from suitable materials, such as TPU filaments. This material should allow the brace to be more compliant around the user's leg and offer some flexibility and more durability to the final product.

A compression sleeve can also be integrated with the brace that allows the spreading of the compression forces.

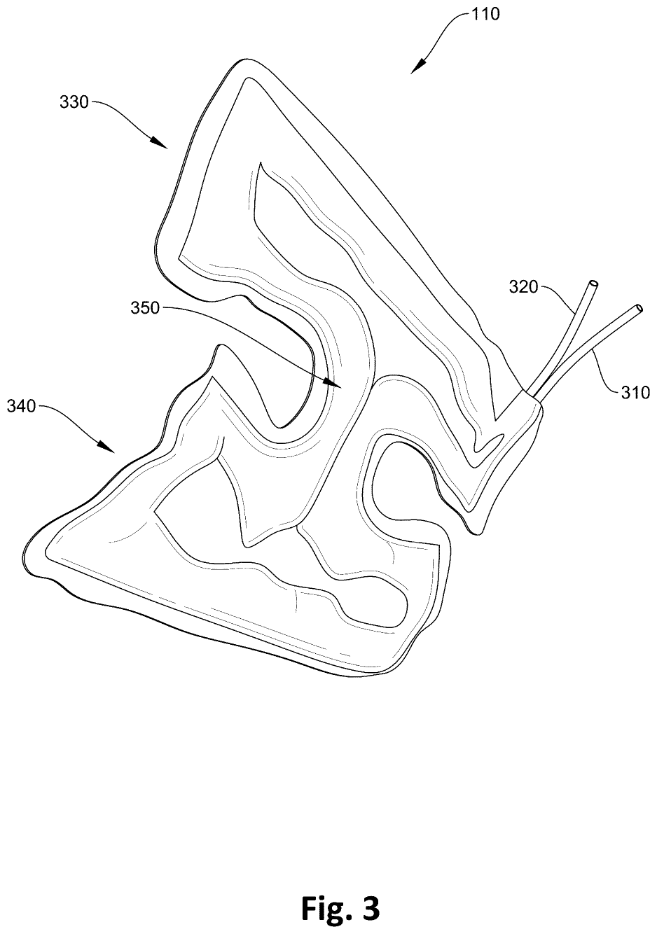

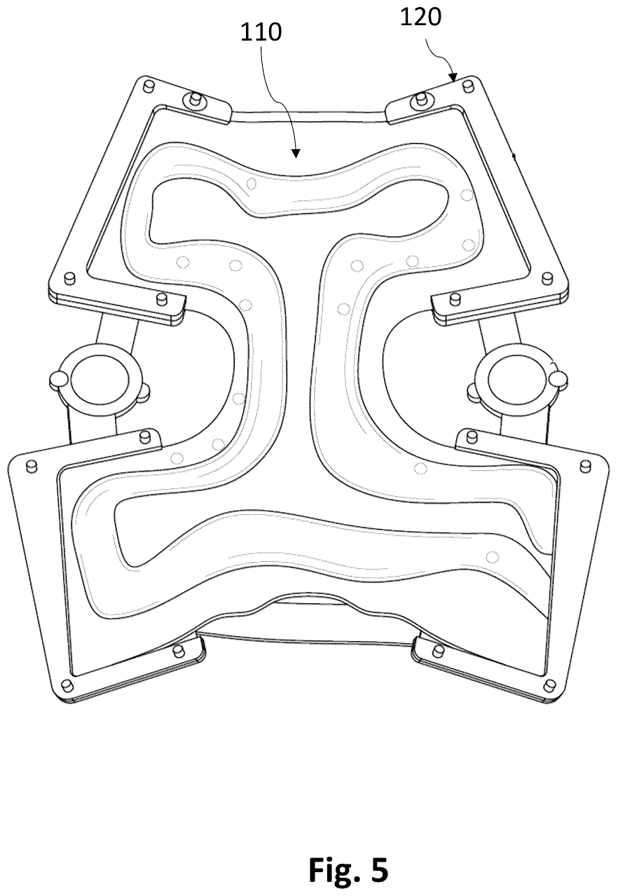

The apparatus may further include a cooling bladder 110 that may be integrated with brace 120 for providing cryotherapy. The cooling bladder may also be separated from the brace. For example, the cooling bladder may be removed for repair or replacement. The cooling bladder shown in FIG. 3 includes an inlet port 310 for receiving a refrigerant liquid, such as chilled water, and an outlet port 320 through which the water can exit, allowing for the circulation of refrigerant fluid through the cooling bladder. The colling bladder as shown in FIG. 3 may include an upper portion 330, a lower portion 340, and a neck 350 extending between the upper portion and the lower portion. The cooling bladder can be coupled to the brace, as shown in FIG. 5. The upper portion 330 of the cooling bladder can be attached to the left and right adapter plates for the upper member of the brace. Similarly, the lower portion of the bladder can be attached to the left and right adapter plates of the lower member of the brace. The top adapter plates can then be attached so that the periphery of the bladder is sandwiched between the top and bottom adapter plates.

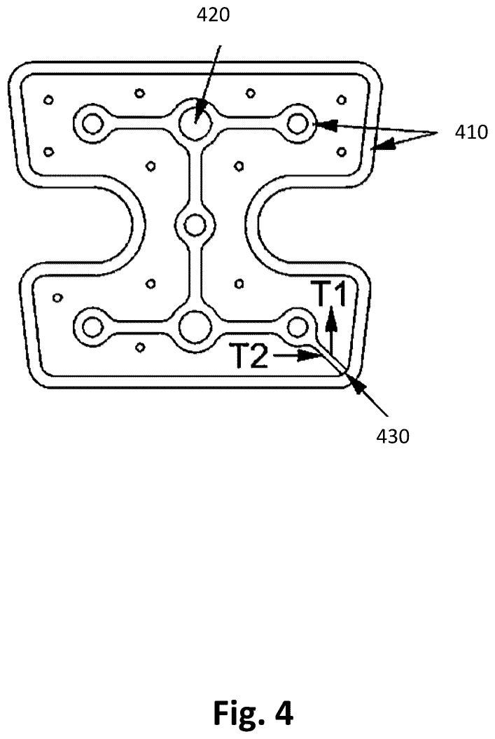

The cooling bladder can be made from suitable materials, such as Nylon or TPU-coated Nylon. The cooling bladder may be in the form of a pouch that includes an upper layer and a lower layer bonded along the periphery. The upper layer and the lower layer can further be bonded at multiple points to prevent the bulging of the cooling bladder. FIG. 4 shows the bonded areas 410 in the cooling bladder. Air cavities 420 can also form in the bonded areas. FIG. 4 also shows a tubing adapter 430 which includes the inlet port and an outlet port. Water through the inlet port can enter an inner volume of the bladder and travel in the direction shown by arrow T1, then flow around the inner volume of the bladder removing heat from the skin, and then can exit through the outlet port along the direction shown by arrow T2.

Each of the inlet tube and the outlet tube can have quick connectors 150 for connecting two main tubing 140 to the chiller 160. The refrigerant liquid, such as water from the water chiller can circulate through the main tubing, the inlet port, the bladder, and the outlet port. Quick connectors allow connecting the main tubing to the chiller when cryotherapy is needed. The quick connectors allow for secure attachment and detachment with minimal effort.

Again, referring to FIG. 5, the cooling bladder may be attached to the brace by attaching the sealed periphery of the bladder to the flange portion of the bottom adapter plates. The periphery of the bladder may align with the periphery of the adapter plates. Suitable adhesives can be used to secure the bladder's periphery to the flange. The top adaptor plates can then be mounted by sandwiching the bladder's periphery between the flanges of the top and bottom adapter plates. In alternative to or in addition to the adhesive, machine screws can be used to fix each bracket. Also, plastic dowl pins, threaded brass inserts, and the like fasteners can be used to secure the two plates of each bracket sandwiching the bladder. The design of the brackets could be optimized for increasing maximum contact of the bladder with skin, or the addition of additional bladders.

In certain implementations, a free-flow cooling bladder, as shown in FIG. 4, with an inlet and outlet ports can be used. It was found that the free-flow cooling bladder provides better heat removal than the tube-type cooling bladder. The water from the inlet can flow around the inner volume of the bladder and exit through the outlet.

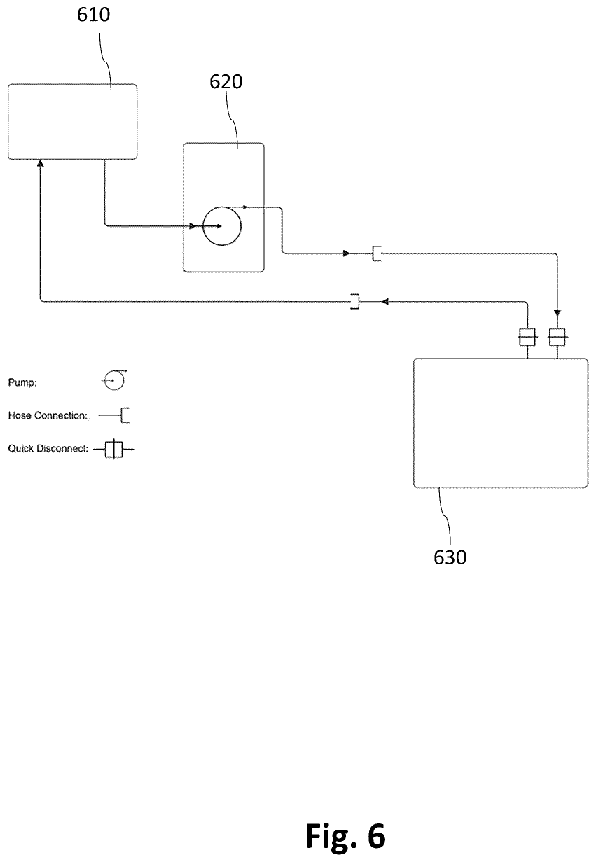

The apparatus may include a water chiller 160. The water chiller includes an auxiliary water tank 162, a heat pump 166, and a water pump 164. FIG. 6 shows a schematic diagram of the water chiller. The heat pump is for cooling the refrigerant liquid, such as water. The structure and functioning of the heat pumps are known in the art. Water can be contained in a chiller tank 610 which can be cooled by the heat pump. The water pump can circulate the water between the chiller tank 610 and the cooling bladder 630. It is understood that any type of cooling liquid can be used, however, it was found that the water chiller provides better cooling efficiency for the cooling bladder followed by the Peltier device.

Auxiliary tank 620 may be used to eliminate any air pockets in the circulation of the water. Coolant can be added to the auxiliary tank. The auxiliary tank can be strategically positioned at the highest point in the system to facilitate the release of trapped air during operation, ensuring continuous and efficient water flow.

The apparatus may include a control unit 170 for controlling the operation of the apparatus. Control unit 170 may include a controller 174 or a microcontroller that can process the instructions. The controller can control the temperature and duration of the cryotherapy. The control unit can include a temperature sensor 178 that can detect the temperature of the refrigerant liquid. The input from the temperature sensor may be used to control the temperature. The control unit may be connected to the heat pump 166 to achieve the desired temperature of the cooling liquid. Display 176 may display the status of the apparatus. For example, the temperature and duration of cryotherapy may be presented on the display. The control unit may also allow connections to an external computing device, such as a smartphone, laptop, computer, and the like. The control unit may receive instructions from an external computing device. Examples of such instructions include temperature, duration, and turning the cooling unit on and off. For example, from a smartphone, the user can set the temperature and duration to start cryotherapy. The control unit may include a beeper to notify the status of the apparatus. For example, on completion of the preset duration, the apparatus may trigger the beeper to inform the user that the cryotherapy has finished.

The control unit may be programmed to effectively manage water levels relative to the bladder to prevent overfilling or underfilling, ensuring optimal performance. The control unit may allow for precise flow control of water. The control mechanisms should be in place to regulate the flow of water through the water chiller and bladder, maintaining desired temperatures and pressures. The Quick-connect and disconnect features facilitate easy installation and maintenance procedures, minimizing downtime. A reliable method for removing air pockets from the bladder and hoses is crucial to prevent disruptions in water flow and system efficiency. Flow control is achieved through the integration of valves and pumps, managed by the control unit. The pump facilitates circulation through the water chiller as well as the bladder. The motor speed is managed to ensure that a consistent flow rate is maintained throughout the bladder and chiller to provide optimal cooling for the patient.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. An apparatus for supporting a body part and cryotherapy, the apparatus comprises:
a brace for supporting the body part, wherein the brace is configured to wrap around the body part to limit movement of the body part; and
a cooling bladder coupled to the brace, wherein the cooling bladder is configured to receive cooling liquid for the cryotherapy.

2. The apparatus of claim 1, wherein the body part is a knee, and the brace is a knee brace.

3. The apparatus of claim 1, wherein the brace comprises an upper member and a lower member, the upper member comprises an upper left bracket and an upper right bracket, the lower member comprises a lower left bracket and a lower right bracket, wherein the cooling bladder is attached to the upper right bracket, the upper left bracket, the lower left bracket, and the lower right bracket.

4. The apparatus of claim 3, wherein the upper left bracket and the lower left bracket are connected through a left hinge joint, and the upper right bracket and the lower right bracket are connected through a right hinge joint.

5. The apparatus of claim 4, wherein the upper left bracket and the upper right bracket are connected through a first strap, wherein the lower left bracket and the lower right bracket are connected through a second strap.

6. The apparatus of claim 5, wherein each of the upper left bracket, the upper right bracket, the lower left bracket, and the lower right bracket comprises a top adapter plate and a bottom adapter plate, wherein portions of the cooling bladder are sandwiched between the top and bottom adapter plates of each of the upper left bracket, the upper right bracket, the lower left bracket, and the lower right bracket.

7. The apparatus of claim 6, wherein the cooling bladder comprises an inlet port, an internal volume, and an outlet port, wherein the inlet port and the outlet port are in fluid communication through the internal volume such that the cooling liquid is received through the inlet port, circulates through the internal volume, and exits from the outlet port.

8. The apparatus of claim 7, wherein the apparatus further comprises a cooling unit, wherein the inlet port and the outlet port of the cooling bladder are configured to fluidly couple to the cooling unit through tubing, wherein the cooling unit is configured to cool the cooling liquid and circulate the cooling liquid through the cooling bladder.

9. The apparatus of claim 8, wherein the apparatus further comprises a control unit configured to control the cooling unit for cryotherapy.

10. The apparatus of claim 9, wherein the body part is a knee, and the brace is a knee brace, wherein the upper member is configured to secure around a thigh, and the lower member is configured to secure around a calf.

11. A method for supporting a body part and providing cryotherapy to the body part while supporting the body part, the method comprises:
providing a brace configured to wrap around the body part for supporting the body part by limit movement of the body part; and
coupling a cooling bladder to an inner side of the brace, wherein the cooling bladder is configured to receive cooling liquid for the cryotherapy.

12. The method of claim 11, wherein the body part is a knee, and the brace is a knee brace, wherein the method further comprises:
wearing the brace integrated with the cooling bladder over the knee;
securing the knee brace; and
circulating the cooling liquid through the cooling bladder for a predetermined duration.

13. The method of claim 11, wherein the brace comprises an upper member and a lower member, the upper member comprises an upper left bracket and an upper right bracket, the lower member comprises a lower left bracket and a lower right bracket, wherein the cooling bladder is coupled to the upper left bracket, upper right bracket, the lower left bracket, and the lower right bracket.

14. The method of claim 13, wherein the upper left bracket and the lower left bracket are connected through a left hinge joint, and the upper right bracket and the lower right bracket are connected through a right hinge joint.

15. The method of claim 14, wherein the upper left bracket and the upper right bracket are connected through a first strap, wherein the lower left bracket and the lower right bracket are connected through a second strap.

16. The method of claim 15, wherein each of the upper left bracket, the upper right bracket, the lower left bracket, and the lower right bracket comprises a top adapter plate and a bottom adapter plate, wherein the method comprises:
sandwiching portions of the cooling bladder between the top and bottom adapter plates of each of the upper left bracket, the upper right bracket, the lower left bracket, and the lower right bracket for coupling the cooling bladder to the brace.

17. The method of claim 16, wherein the cooling bladder comprises an inlet port, an internal volume, and an outlet port, wherein the inlet port and the outlet port are in fluid communication through the internal volume such that the cooling liquid is received through the inlet port, circulates through the internal volume, and exits from the outlet port.

18. The method of claim 17, wherein the method further comprises:
coupling the inlet port and the outlet port of the cooling bladder to a cooling unit through tubing, wherein the cooling unit is configured to cool the cooling liquid and circulate the cooling liquid through the cooling bladder.

19. The method of claim 18, wherein the method further comprises:
coupling the cooling unit to a control unit, wherein the control unit is configured to control the cryotherapy.

20. The method of claim 19, wherein the body part is a knee, and the brace is a knee brace, wherein the method further comprises:
securing the upper member around a thigh;
securing the lower member around a calf;
connecting the cooling bladder to the cooling unit through the tubing; and
circulating the cooling liquid through the cooling bladder for a predetermined duration for the cryotherapy; and
after the predetermined duration, turn off the cooling unit and disconnecting the cooling bladder from the cooling unit.
